(19) United States
(12) Patent Application Publication
(10) Pub. No.: US 2002/0197311 A1
(43) Pub. Date: Dec. 26, 2002

(54) Pharmaceutical preprations containing pyrogenic silicon dioxide

(75) Inventors: Steffen Hasenzahl, Hanau (DE); Margarete Drechsler, Gelnhausen (DE); Michael Hirschhaeuser, Usingen-Eschbach (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Degussa AG, Duesseldorf (DE)

(21) Appl. No.: 10/157,181

(22) Filed: May 30, 2002

(30) Foreign Application Priority Data
May 30, 2001 (DE) 101 26 163.2

(51) Int. Cl. A61K 31/60; A61K 9/20; A61K 31/19; A61K 9/14; A61K 31/192

(52) U.S. Cl. 424/465; 514/165; 514/570; 514/629

(57) ABSTRACT

Pharmaceutical and cosmetic preparations containing pyrogenic silicon dioxide with a tamped density of 80-250 g/l. Preparations and compositions as solid, semisolid and liquid forms. Products, such as cosmetics, and methods of treatment using such compositions.

### CROSS-REFERENCE TO RELATED APPLICATIONS

This application claims priority to DE (Germany) 101 26 163.2, filed May 30, 2001, which is hereby incorporated by reference in its entirety.

### BACKGROUND OF THE INVENTION

1. Field of the Invention

Products and compositions, such as pharmaceuticals and cosmetics, containing pyrogenic silicon dioxide with a tamped density of 80 to 250 g/l. Methods for making such products. These products have a number of superior properties, including improved flowability or mechanical stability, and decreased dust production.

2. Description of the Related Art

A drug normally comprises two groups of substances with different functions, namely active ingredients and auxiliary substances or excipients. The behaviour of the final product, such as a drug mixture or cosmetic, depends on process variables and on the interrelationship of the active ingredient(s) and the excipient(s).

There is a continuing need to develop new excipient mixtures or new physical forms of excipients that further improve the safety, handling, and processing of pharmaceutical and cosmetic products, as well as the physical and functional characteristics of the final commercial products.

Active ingredients are characterised by their specific pharmacological action. They represent the active constituent of a drug. As such, they are identified quantitatively on both the packaging and the package insert.

Auxiliary substances or excipients, on the other hand, generally exert no significant pharmacological action. However, they are required so that the active ingredient or drug may be prepared in a suitable form, for instance, in a form suitable for administration by a particular route. Excipients may also be added to improve handling of a product, such as a pharmaceutical, during its manufacture, or to improve the stability of the active component of such a pharmaceutical. A pharmaceutical product normally contains several auxiliary substances with different functions, such as fillers, binders, disintegrants, lubricants or release agents.

A large number of auxiliary substances can be utilised for the development of stable, easy-to-use and effective drugs comprising one or more active ingredient(s) and/or auxiliary substance(s).

Highly dispersed, pyrogenic silicon dioxide, for example Aerosil®, is often used in pharmaceutical and cosmetic preparations. It can be used as a free flow agent, adsorbent and desiccant in solid product forms and as a suspension stabiliser and gelling agent in liquid and semi-liquid product forms. It can also be used to increase the mechanical stability and the disintegration rate of tablets and can improve the distribution of the active ingredient in a pharmaceutical or cosmetic preparation. On the other hand, highly dispersed, pyrogenic silicon dioxide can also act as an active ingredient, for example, for the treatment of gastritis, enteritis or skin bums see No. 49 of technical bulletin pigments, “AEROSIL in Pharmaceuticals and Cosmetics, Degussa AG) which is incorporated by reference.

The preparation of pharmaceutical and cosmetic products demands a very high level of cleanliness. One particular disadvantage of working with highly dispersed silicon dioxide is the production of dust during the preparation of such products. Production of dust also represents a potential environmental, occupational or health risk, especially for workers in production or manufacturing facilities.

The highly dispersed silicon dioxide in conventional use is also difficult to handle and process due to its low bulk density and low tamped density. Consequently, the time and effort required to produce pharmaceutical and cosmetic preparations is considerably increased, thus lowering production efficiency and increasing costs.

The identification of an excipient that improves the flowability of mixtures used in the preparation of pharmaceutical and cosmetic products would also be highly advantageous to further improve efficiency and economy of such processes, improve ingredient dispersion and achieve higher dosage accuracy in the production of products, such as capsules and tablets. For instance, it would be highly desirable to achieve a lower variance in tablet and capsule weights, while facilitating the production of such products and reducing production costs.

### BRIEF DESCRIPTION OF THE INVENTION

One object of the present invention is to provide pharmaceutical and cosmetic preparations that avoid the disadvantages involved with using existing excipients, such as conventional forms of pyrogenic silicon dioxide.

The object is achieved by producing and using pharmaceutical and cosmetic preparations containing, as an auxiliary substance pyrogenic silicon dioxide with a tamped density of about 80 to about 250 g/l, determined according to DIN 55943.

This result is surprising because it could not be assumed that the properties of pharmaceutical and cosmetic preparations, for example, dust production, flowability or mechanical stability, would be influenced by the tamped density of the pyrogenic silicon dioxide.

It has been found that, when working with the preparations according to the invention, only slight dust production occurs and the flowability of the preparations is markedly higher than that of state-of-the-art preparations. In addition, it has been found that the mechanical stability of tablets formulated with such preparations is improved and the capsule weight is increased.

It has been found to be particularly favourable to choose the tamped density of the silicon dioxide between 100 and 200 g/l. Other tamped density ranges including 100-120, 120-140, 140-160, 160-180 or 180-200 g/l as well as any specific intermediate value, e.g. 105, 106, 107, 110, 115, 125, 130, 135, 145, 150, 155, 156, 165, 166, 170, 190, 195 g/l, etc. are also contemplated. However, a highly dispersed silicon dioxide with a tamped density of about 120 g/l is particularly preferred.

Furthermore, the inventors have also found that it is advantageous to choose pyrogenic silicon dioxide with a BET specific surface area of 50 to 400 m²/g or any specific intermediate value within this range, determined according to DIN 66131. For instance, ranges of 50-75, 75-100, 100-125, 125-150, 150-175, 175-200, 200-225, 225-250, 250-275, 275-300, 300-325, 325-350, 350-375, and 375-400 m²/g are contemplated. A BET specific surface area of 90-250 m²/g is particularly advantageous.

Based on the above findings, an appropriate amount of pyrogenic silicon dioxide having a tamped density of about 80 to about 250 g/l for incorporation into a preparation may be selected by one with skill in the art. However, the inventors have found that the amount of pyrogenic silicon dioxide in the preparation according to the invention is preferably 0.1 to 10 wt. %, or any intermediate value within this range, such as 0.15, 0.2, 0.3, 0.4, 0.5, 0.6, 0.7, 0.8, 0.9, 1, 1.1, 1.5,2,2.5,3,4, 5,6,7, 7.5, 8,9,9.5 or 9.9 wt %.

In addition, the preparation according to the invention can contain the conventional auxiliary substances used in pharmaceuticals or cosmetics, for example, fillers such as carbohydrates, sugar alcohols, starch and starch derivatives; binders such as gelatine, cellulose and polyvinylpyrrolidone derivatives; disintegrants such as carboxymethyl cellulose, corn starch and sodium carboxymethyl starch; intestinal lubricants such as talcum or polyethylene glycols; and mould lubricants and release agents such as magnesium or calcium stearate or stearic acid.

Other products, especially those prepared in tablet or capsule form, or those in which improved flowability is required during processing or production, may also incorporate as an auxiliary substance pyrogenic silicon dioxide with a tamped density of about 80 to about 250 g/l. For instance, the flowability of food products, industrial or commercial products or products containing chemically or biologically active ingredients, especially those in powdered form, can be improved by the addition of pyrogenic silicon dioxide with a tamped density of about 80 to about 250 g/l. Moreover, commercial or industrial products may also contain pyrogenic silicon dioxide with a tamped density of about 80 to about 250 g/l to improve their physical properties, such as mechanical stability, hardness, product weight, dispersion of ingredients or to provide better accuracy in their formulation. Examples of such products include solid products such as tablets containing surfactants, such as bath tablets, laundry tablets containing detergents, chelators, or enzymatic cleaners, cleaners, including bleaches and household cleaners, tablets for addition to water, such as to pH pool water or containing chlorine or other biocides, tablets containing enzymes, tablets containing pigments or dyes, tablets containing indicators for use in assays or diagnostic kits, tablets containing dispersible foods or nutritional products, or solid forms or tablets containing other chemically or biologically active subtances. Solid or powdered forms of cleaning agents, enzymes, dyes, minerals, fertilisers, herbicides, or pesticides may also contain pyrogenic silicon dioxide with a tamped density of about 80 to about 250 g/l.

Processes for the preparation of pyrogenic silicon dioxide can be found for example in Ullmann's Encyclopedia of Industrial Chemistry, vol. A23, page 635 et seq., 5th edition, 1993, which is hereby incorporated by reference.

Pyrogenic silicon dioxide also includes doped oxides and mixed oxides in which the silicon dioxide content is at least 90%. Doped pyrogenic silicon dioxides can be obtained, for example, by the process described in DE-A-196 50 500, the doping being incorporated via an aerosol from a salt solution or suspension into a flame such as that used for the preparation of pyrogenic oxides. A mixed oxide with a silicon dioxide content of more than 90 wt. % can be obtained, for example, by the process described in DE-A-199 19 635.

Furthermore, all or part of the surface of the silicon dioxide can be rendered hydrophobic by means of a subsequent treatment with a surface-modifying reagent. Relevant processes are known in the art and can also be found, for example, in DE-A-11 63 784, DE-A-196 16 781, DE-A-197 57 210 or DE-A-44 02 370.

The highly dispersed, pyrogenic silicon dioxide acquires its tamped density either immediately during preparation or in a downstream process step. Thus, for example, compaction processes for pyrogenic silicon dioxide are described in DE-A-32 38 427 and DE-A-37 41 846. Compaction by means of a rotary vacuum filter equipped with a compacted strip, as indicated in DE-A-37 41 846, is particularly advantageous for the preparation according to the invention. Such products, which are marketed, for example under the name AEROSIL 200 VV, are distinguished by a particularly uniform and homogeneous structure.

Mixtures of pyrogenic silicon dioxide with doped silicon dioxide having an SiO₂ content of 90%, with mixed oxides having an SiO₂ content of 90% or more, and/or with silicon dioxide which has been rendered hydrophobic, can also be used for the preparations according to the invention.

The preparation according to the invention can contain highly dispersed, pyrogenic silicon dioxide with a tamped density of between 80 and 250 g/l in combination with any desired pharmaceutical active ingredient. Examples of pharmaceutical active ingredients which may be mentioned are α-proteinase inhibitor, abacavir, abciximab, acarbose, acetylsalicylic acid, acyclovir, adenosine, albuterol, aldesleukin, alendronate, alfuzosin, alosetron, alprazolam, alteplase, ambroxol, amifostine, amiodarone, amisulpride, amlodipine, amoxycillin, amphetamine, amphotericin, ampicillin, amprenavir, anagrelide, anastrozole, ancrod, antihaemophilic factor, aprotinin, atenolol, atorvastatin, atropine, azelastine, azithromycin, azulene, bamidipine, beclomethasone, benazepril, benserazide, beraprost, betamethasone, betaxolol, bezafibrate, bicalutamide, bisabolol, bisoprolol, botulin toxin, brimonidine, bromazepam, bromocriptine, budesonide, bupivacaine, bupropion, buspirone, butorphanol, cabergoline, calcipotriene, calcitonin, calcitriol, camphor, candesartan, candesartan cilexetil, captopril, carbamazepine, carbidopa, carboplatin, carvedilol, cefaclor, cefadroxil, cefazolin, cefdinir, cefepime, cefixime, cefinetazole, cefoperazone, cefotiam, cefoxitin, cefoxopran, cefpodoxime, cefprozil, ceftazidime, ceftibuten, ceftriaxone, cefuroxime, celecoxib, celiprolol, cephalexin, cerivastatin, cetirizine, chloramphenicol, cilastatin, cilazapril, cimetidine, ciprofibrate, ciprofloxacin, cisapride, cisplatin, citalopram, clarithromycin, clavulanic acid, clindamycin, clomipramine, clonazepam, clonidine, clopidogrel, clotrimazole, clozapine, cromolyn, cyclophosphamide, cyclosporin, cyproterone, dalteparin, deferoxamine, desogestrel, dextroamphetamine, diazepam, diclofenac, didanosine, digitoxin, digoxin, dihydroergotamine, diltiazem, diphtheria protein, diphtheria toxoid , divalproex, dobutamine, docetaxel, dolasetron, donepezil, dornase-α, dorzolamide, doxazosin, doxifluridine, doxorubicin, dydrogesterone, ecabet, efavirenz, enalapril, enoxaparin, eperisone, epinastine, epirubicin, eptifibatide, erythropoietin-α, erythropoietin-β, etanercept, ethynyloestradiol, etodolac, etoposide, factor VIII, famciclovir, famotidine, faropenem, felodipine, fenofibrate, fenoldopam, fentanyl, fexofenadine, filgrastim, finasteride, flomoxef, fluconazole, fludarabine, flunisolide, flunitrazepam, fluoxetine, flutamide, fluticasone, fluvastatin, fluvoxamine, follitropin-α, follitropin-β, formoterol, fosinopril, furosemide, gabapentin, gadodiamide, ganciclovir, gatifloxacin, gemcitabine, gestodene, glatiramer, glibenclamide, glimepiride, glipizide, glyburide, goserelin, granisetron, griseofulvin, hepatitis B antigen, hyaluronic acid, hycosin, hydrochlorothiazide, hydrocodone, hydrocortisone, hydromorphone, hydroxychloroquine, hylan G-F 20, ibuprofen, ifosfamide, imidapril, imiglucerase, imipenem, immunoglobulin, indinavir, indomethacin, infliximab, insulin, insulin human, insulin lispro, insulin aspart, interferon-β, interferon-α, iodine-125, iodixanol, iohexol, iomeprol, iopromide,, ioversol, ioxoprolene, ipratropium, ipriflavone, irbesartan, irinotecan, isosorbide, isotretinoin, isradipine, itraconazole, potassium chlorazepate, potassium chloride, ketorolac, ketotifen, whooping cough vaccine, clotting factor IX, lamivudine, lamotrigine, lansoprazole, latanoprost, leflunomide, lenograstim, letrozole, leuprolide, levodopa, levofloxacin, levonorgestrel, levothyroxine, lidocaine, linezolide, lisinopril, lopamidol, loracarbef, loratadine, lorazepam, losartan, lovastatin, lysine acetylsalicylic acid, manidipine, mecobalamin, medroxyprogesterone, megestrol, meloxicam, menatetrenone, meningococcus vaccine, menotropin, meropenem, mesalamine, metaxalone, metformin, methylphenidate, methylprednisolone, metoprolol, midazolam, milrinone, minocycline, mirtazepine, misoprostol, mitoxantrone, moclobemide, modafinil, mometasone, montelukast, morniflumate, morphium, moxifloxacin, mycophenolate, nabumetone, nadroparin, naproxen, naratriptan, nefazodone, nelfinavir, nevirapine, niacin, nicardipine, nicergoline, nifedipine, nilutamide, nilvadipine, nimodipine, nitroglycerin, nizatidine, norethindrone, norfloxacin, octreotide, olanzapine, omeprazole, ondansetron, orlistat, oseltamivir, oestradiol, oestrogens, oxaliplatin, oxaprozin, oxolinic acid, oxybutynin, paclitaxel, palivizumab, pamidronate, pancrelipase, panipenem, pantoprazole, paracetamol, paroxetine, pentoxifylline, pergolide, phenytoin, pioglitazone, piperacillin, piroxicam, pramipexole, pravastatin, prazosin, probucol, progesterone, propafenone, propofol, propoxyphene, prostaglandin, quetiapin, quinapril, rabeprazole, raloxifene, ramipril, ranitidine, repaglinide, reserpine, ribavirin, riluzole, risperidone, ritonavir, rituximab, rivastigmin, rizatriptan, rofecoxib, ropinirole, rosiglitazone, salmeterol, saquinavir, sargramostim, serrapeptase, sertraline, sevelamer, sibutramine, sildenafil, simvastatin, somatropin, sotalol, spironolactone, stavudine, sulbactam, sulfaethidole, sulfamethoxazole, sulfasalazine, sulpiride, sumatriptan, tacrolimus, tamoxifen, tamsulosin, tazobactam, teicoplanin, temocapril, temozolomide, tenecteplase, tenoxicam, teprenone, terazosin, terbinafine, terbutaline, tetanus toxoid, tetrabenazine, tetrazapam, thymol, tiagabine, tibolone, ticarcillin, ticlopidine, timolol, tirofiban, tizanidine, tobramycin, tocopheryl nicotinate, tolterodin, topiramate, topotecan, torasemide, tramadol, trandolapril, trastuzumab, triamcinolone, triazolam, trimebutine, trimethoprim, troglitazone, tropisetron, tulobuterol, unoprostone, urofollitropin, valacyclovir, valproic acid, valsartan, vancomycin, venlafaxine, verapamil, verteporfin, vigabatrin, vinorelbine, vinpocetine, voglibose, warfarin, zafirlukast, zaleplon, zanamivir, zidovudine, zolmitriptan, zolpidem, zopiclone and derivatives thereof. However, pharmaceutical active ingredients are also to be understood as including other substances such as vitamins, provitamins, antioxidants, enzymes, amino acids, proteins or protein supplements, minerals or mineral supplements, anabolic or metabolic supplements or aids, health, weight loss or dietary supplements, essential fatty acids, extracts of vegetable and animal origin, herbs and herb extracts, fiber supplements, probiotic organisms, and oils of vegetable and animal origin.

The pharmaceutical compositions in which highly dispersed, pyrogenic silicon dioxide can be used also include plant-based pharmaceutical preparations and homeopathic preparations.

The pharmaceutical preparations according to the invention can also be so-called sustained action and depot forms with controlled release of the active ingredient. The pharmaceutical preparations according to the invention can also be part of therapeutic systems, for instance Do therapeutic systems for local application and transdermal therapeutic systems.

In one advantageous embodiment, the preparations according to the invention contain paracetamol, acetylsalicylic acid or ibuprofen as the active ingredient.

The preparations according to the invention can be any solid, semisolid or liquid pharmaceutical form, preferably for oral and/or topical administration, e.g. in suspensions, emulsions, aerosols, ointments, creams, gels, pastes, suppositories, sticks, powders, granules, tablets, lozenges, coated tablets, film-coated tablets, hard gelatine capsules, soft gelatine capsules, extrudates, microcapsules or microspheres. Solid pharmaceutical forms are particularly preferred, examples being powders, granules, tablets and capsules. The expression “pharmaceutical composition” in terms of the present invention also includes precursors and intermediates for the production of granules, tablets, capsules, suspensions, inspissated juices and inspissated drops. Such precursors and intermediates can take the form of a powder, granules or an extrudate, for example.

Methods of producing solid, semisolid and liquid pharmaceutical forms are known and are described in numerous publications and textbooks on pharmaceutical technology; cf., for example, K. H. Bauer, K.-H. Frömming, C. Führer, Lehrbuch der pharmazeutischen Technologie (Textbook of Pharmaceutical Technology), 6th edition, Wissenschaftliche Verlagsgesellschaft mbH, Stuttgart (1999), which is hereby incorporated by reference.

### EXAMPLES

Preparations:

The pulverulent ingredients are weighed out to an accuracy of 0.01 g in the order indicated and are mixed by hand in a glass flask. This mixture is passed through a sieve of mesh size 0.75 mm and homogenised in a glass flask for five minutes with a turbula mixer. Flowability for each preparation is shown below in Tables 2 and 3, respectively.

TABLE 1
Preparations (data in wt. %)

| | Preparation 1 | Preparation 2 |
|---|---|---|
| Paracetamol | 83.3 | — |
| Acetylsalicylic acid | — | 83.3 |
| Powdered cellulose | 13.3 | 10.4 |
| Corn starch | 3.0 | 5.0 |
| Magnesium stearate | 0.1 | — |
| Stearic acid | — | 1.0 |
| Silicon dioxide | 0.3 | 0.3 |

The preparations shown in Table 1 are then compressed into tablets and filled into capsules.

Hard Gelatine Capsules.

Using a capsule filling apparatus, size 1 hard gelatine capsules with an empty weight of 71-78 mg were filled with the preparations in Table 1. 60 capsules of each preparation are produced and the mean capsule weight was determined. Tables 2 and 3, below, show the values (capsule weight) for Preparations 1 and 2, respectively.

Tablets

Using an eccentric press, the preparations in Table 1 were compressed under the same pressure to tablets weighing approx. 600 mg. The tablet hardness was determined on 10 tablets of each preparation by means of a semiautomatic hardness tester. The values (tablet hardness) for Preparations 1 and 2 are shown below in Tables 2 and 3 respectively.

TABLE 2
Properties of Preparation 1

| Tamped density of | | | |
|---|---|---|---|
| SiO₂ | | Tablet hardness | Capsule weight |
| [g/l] | Flowability* | [N] | [mg] |
| 50 | good to | 116 | 368 |
| | satisfactory | | |
| 75 | good to | 115 | 371 |
| | satisfactory | | |
| 120 | very good | 138 | 392 |
| 150 | very good | 140 | 391 |

*Flowability determined according to: no. 11 of publication series on pigments, 6th edition, Degussa.

TABLE 3
Properties of Preparation 2

| Tamped density of | | | |
|---|---|---|---|
| SiO₂ | | Tablet hardness | Capsule weight |
| [g/l] | Flowability* | [N] | [mg] |
| 50 | good | 86 | 367 |
| 75 | good | 91 | 365 |
| 120 | very good | 130 | 380 |
| 150 | very good | 134 | 385 |

As shown above, preparations containing silicon dioxide having a tamped density between about 80 about 250 g/l clearly show advantages in terms of flow behaviour, tablet hardness and capsule weight.

### Modifications and Other Embodiments

Various modification and variations of the described compositions and their methods of use as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the pharmaceutical, cosmetic, nutritional, medical, biological, chemical or related fields are intended to be within the scope of the following claims. Obviously, numerous modifications and variations of the present invention are possible in light of the above teachings. It is therefore to be understood that within the scope of the appended claims, the invention may be practiced otherwise than as specifically described herein.

### Incorporation by Reference

Each document, patent, patent application or patent publication cited by or referred to in this disclosure is incorporated by reference in its entirety. However, no admission is made that any such reference constitutes prior art and the right to challenge the accuracy and pertinence of the cited documents is reserved. Specifically, priority document DE (Germany) 101 26 163.2, filed May 30, 2001 is hereby incorporated by reference.

What is claimed as new and is intended to be secured by Letters Patent is:

1. A composition comprising:
pyrogenic silicon dioxide, wherein said silicon dioxide has a tamped density ranging from about 80 to about 250 g/l and one or more drugs or pharmaceutically active compounds.

2. The composition of claim 1, wherein said silicon dioxide has a tamped density ranging from about 100 to about 200 g/l.

3. The composition of claim 1, comprising silicone dioxide that has a BET specific surface area between about 50 and about 400 m²/g.

4. The composition of claim 1 that has a BET specific surface area between about 90-250 m²/g.

5. The composition of claim 1 comprising about 0.1 to about 10 wt. % of pyrogenic silicon dioxide.

6. The composition of claim 1, wherein said pyrogenic silicon dioxide comprises a mixed oxide with a silicon dioxide content of 90 wt. % or more.

7. The composition of claim 1, wherein said pyrogenic silicon dioxide comprises a doped oxide with a silicon dioxide content of 90 wt. % or more.

8. The composition of claim 1, wherein the surface of the pyrogenic silicon dioxide has been rendered hydrophobic.

9. The composition of claim 1, comprising paracetamol, acetylsalicylic acid or ibuprofen, or a combination of two or more thereof.

10. The composition of claim 1, further comprising one or more excipient(s), auxiliary substance(s) or carrier(s).

11. The composition of claim 1 in the form of a solid.

12. The composition of claim 1 in semi-solid form.

13. The composition of claim 1 in liquid form.

14. The composition of claim 1 in the form of a powder.

15. The composition of claim 1 in the form of a granule.

16. The composition of claim 1 in the form of a capsule.

17. The composition of claim 9 in the form of a hard or soft gelatin capsule.

18. The composition of claim 1 in the form of a tablet.

19. The composition of claim 11 in the form of a coated tablet or a film-coated tablet.

20. The composition of claim 1 in the form of a suspension, emulsion, lotion, aerosol, ointment, cream, gel, paste, suppository, stick, lozenge, extrudate, microcapsule or microsphere.

21. A composition comprising:
pyrogenic silicon dioxide, wherein said silicon dioxide has a tamped density ranging from about 80 to about 250 g/l and one or more cosmetic ingredient(s).

22. A composition comprising:
pyrogenic silicon dioxide, wherein said silicon dioxide has a tamped density ranging from about 80 to about 250 g/l and one or more more nutritionally, chemically or biologically active ingredient(s).

23. The composition of claim 22 comprising one or more vitamin(s) or nutrient(s).

24. The composition of claim 22, further comprising one or more surfactants.

25. The composition of claim 22, further comprising one or more enzyme(s) or chelating agent(s).

26. A method for making a pharmaceutical or cosmetic product comprising admixing one or more pharmaceutically active compound(s) or cosmetic ingredients with pyrogenic silicon dioxide, wherein said silicon dioxide has a tamped density of about 80 to about 250 g/l.

27. The method of claim 26, comprising pyrogenic silicone dioxide that has a BET specific surface area between about 50 and about 400 m²/g.
